(19) United States
(12) Patent Application Publication
(10) Pub. No.: US 2004/0198698 A1
(43) Pub. Date: Oct. 7, 2004

(54) Nitrate ester-cyclodextrin complexes

(76) Inventors: Walter Heinzelmann, Odenthal (DE); Stephan Bojar, Windhagen (DE); Cornelius Ruloff, Leverkusen (DE)

Correspondence Address: FULBRIGHT & JAWORSKI, LLP, 666 FIFTH AVE, NEW YORK, NY, 10103-3198, US

(21) Appl. No.: 10/483,219

(22) PCT Filed: Jul. 19, 2002
(86) PCT No.: PCT/DE02/02666

(30) Foreign Application Priority Data
Jul. 20, 2001 (DE) 101 34 683.2

(51) Int. Cl. A61K 31/724; C08B 37/16

(52) U.S. Cl. 514/58; 536/46

(57) ABSTRACT

The invention relates to 1:1 complexes of organic nitrate esters with cyclodextrins, to the production of these complexes and to the use thereof in the field of coronary medicine.

The present invention relates to 1:1 complexes of organic nitrate esters with cyclodextrins, the preparation of these complexes and the use of the latter in coronary medicine.

α-, β- and γ-cyclodextrins are known complex-forming agents for the formation of cyclodextrin inclusion compounds, in particular based on the preferably used β-cyclodextrins. α-, β- and γ-cyclodextrins form water-soluble complexes with a fairly large number of organic and inorganic compounds as well as with solvents and water as guest molecules, the complexes being of great interest for technical, cosmetic or pharmaceutical and medical applications. For this purpose the internal hydrophobic or external hydrophilic cavity configuration of the cyclodextrins is optionally modified by the introduction of additional substituents in order to alter their chemical and/or physical behaviour in the complex formation.

Thus, β-cyclodextrin complexes with potentially explosive, liquid or solid nitrate esters, such as for example with glycerol trinitrate (GTN), or with isosorbide dinitrate (ISDN) and 5-isosorbide mononitrate (ISMN) have become known for possible applications in coronary medicine. On account of their stereometric dimensions and/or structure these active substances are able, as for example in the case of GTN, to form 1:1 complexes with β-cyclodextrin. On the other hand incompatible size relationships such as exist for example in the similarly coronary-active pentaerythritol tetranitrate (PETN) with respect to β-cyclodextrin, did not lead to the formation of β-cyclodextrin complexes.

Galenically advantageous therapeutic effects can be achieved by the controlled release of these effective vasodilating short-acting nitrate ester active substances such as GTN or the longer-acting nitrate ester active substances ISDN and ISMN from their water-soluble β-cyclodextrin complexes. However, a particular factor in this case is that, due to inclusion of each individual molecule of the aforementioned potentially explosive and mechanically, thermally and chemically in some cases extremely sensitive active substances such as GTN, an optimal desensitisation as regards the danger of explosion is achieved. This is manifested in that, inter alia, the complexed active substances are thermally substantially more stable and the medicaments produced therefrom can be stored for a longer period.

β-cyclodextrin inclusion compounds with liquid or solid nitrate esters have been prepared in a known manner as 1:1 complexes with for the most part defined compositions by for example reacting aqueous solutions or also slurries of β-cyclodextrin in water with solutions of the nitrate ester in organic solvents with an active substances concentration adjusted to the corresponding β-cyclodextrin concentration, under stirring and metering in of the active substances solutions. As organic solvents there have preferably been used methanol or ethanol, or, depending on the water solubility of the nitrate ester, also water. The reaction was carried out at temperatures in the range from 40° to 80° C. Following this the resultant complexes crystallised out under controlled cooling of the mixture over a relatively long time to room temperature. The complexes were separated with organic solvents and then washed and dried by heating.

As practical experience has shown however, particularly when handling liquid nitrate esters such as the potentially explosive compound GTN, the preparation of the desired 1:1 complexes with the active substance GTN does not proceed without problems: for example, when metering a solution of GTN in water-soluble, organic solvents such as methanol or ethanol into an aqueous solution of β-cyclodextrin (β-CD) while stirring at temperatures of 40° to 70° C. followed by slow and controlled cooling of the reaction mixture to room temperature (20° C.), the GTN/β-CD complexes that crystallised out were obtained only in a moderate and relatively variable yield. In this connection the re-use of the mother liquors also did not produce any significant improvement. Thus, for example, the GTN content of the complexes obtained after drying was as a rule significantly below the theoretical required content of 16.67 wt. % GTN for a GTN/β-CD (1:1) complex.

This is in part attributable to the fact that in the relatively slowly proceeding complex formation, a plurality of guest molecules (water, organic solvents, GTN) compete with one another within the framework of temperature-dependent equilibria, with the result that in the reaction product the theoretical GTN content of the desired GTN/β-CD (1:1) complex is not fully achieved. Further very substantial GTN losses may occur due to the use of the solvents conventionally employed for washing the separated reaction product, the solvents being chosen for example from alcohols (methanol, ethanol, isopropanol), ethers (diethyl ether), ketones (acetone) or esters (ethyl acetate). Although these are good solvents for removing still adhering GTN, they may however also in turn completely wash out the included active substance GTN from the inclusion complexes, particularly with continued use.

In the preparation, irrespective of the preparation method excesses of GTN, that can no longer be absorbed by the equimolar-calculated weighed-out amount of β-CD and which can lead to safety problems, should always be avoided. Thus excess GTN, which is insoluble in water, can accumulate in the mother liquor or, after cooling the reaction mixture down to below 15° C. (melting point 13.2°-13.5°C.) may be precipitated as a particularly sensitive solid together with the reaction product.

The object of the present invention was accordingly to prepare a 1:1 complex of an organic nitrate ester with a cyclodextrin with an approximately theoretical content of organic nitrate ester, without the organic nitrate ester having to be used in excess referred to the employed cyclodextrin.

The object on which the invention is based is solved by a 1:1 complex of an organic nitrate ester with a cyclodextrin, having the features of the main claim. Preferred modifications are characterised in the subclaims.

Surprisingly it was found in fact that the comparatively best yields of a 1:1 complex of an organic nitrate ester with a cyclodextrin with an approximately theoretical content of organic nitrate ester are obtained if asymmetrically structured ethers, for example tert.-butyl methyl ether (TBME) with the linearised structural formula H₃C—O—C(CH₃)₃, are used for washing the separated reaction product. Being a readily volatile solvent, this removes externally adhering organic nitrate ester without leaving a residue, butin contrast to conventional solvents is however not able to wash out complexed organic nitrate esters.

The 1:1 complex according to the invention may be obtained for example by the following procedure carried out under appropriate safety precautions:

### EXAMPLE

After completely dissolving 73.6 g of dry β-cyclodextrin by stirring in 1260 ml of water at 50° C., 15.4 g of pure GTN are added dropwise from a suitable metering device to the resultant clear solution within 20-30 minutes without using auxiliary solvents, while stirring with a cavitation-free turbine stirrer running at ca. 500 rpm in the 50° C. hot solution, and emulsified. Following this the solution becomes turbid after a relatively short time due to the incipient crystallisation of the GTN/β-CD complex. After completing the addition of GTN the reaction mixture is stirred for a further 1 to 1.5 hours at 50° C. and is then slowly cooled to room temperature (20° C.) over a period of 2.5 to 3 hours while stirring. The solid obtained is suction-filtered and washed, firstly with mother liquor and then three times with 50 ml of TBME each time, and then dried at 48°-49° C. The dry yields are 85-90% of theory. The GTN content is on average 16.50% GTN (theory: 16.67% GTN), which corresponds to 99% of theory.

1. A 1:1 of an organic nitrate ester with a cyclodextrin wherein the complex contains more than 95%, preferably more than 98% of the theoretical required content of organic nitrate ester.

2. The complex according to claim 1, wherein the organic nitrate ester is selected from the group comprising glycerol trinitrate, isosorbide dinitrate and isosorbide-5-mononitrate.

3. The complex according to claim 1, wherein the organic nitrate ester is glycerol trinitrate (GTN).

4. The complex according to claim 1, wherein the cyclodextrin is selected from the group comprising α-cyclodextrin, β-cyclodextrin, and γ-cyclodextrin.

5. The complex of claim 1, wherein cyclodextrin is β-cyclodextrin.

6. A process for the preparation of a complex according to claim 1, comprising the steps of:
dissolution of the cyclodextrin at elevated temperature in water,
addition of the organic nitrate ester,
cooling to room temperature,
isolation of the solid,
washing the solid with an asymmetric ether,
drying the complex.

7. The process according to claim 6, wherein only pure organic nitrate ester (100%) without the use of auxiliary solvents is employed for the preparation of the complex.

8. The process according to claim 6 wherein the preparation of the complex is carried out at 40°-80° C., preferably at 40°-60° C., particularly preferably at 50° C.

9. The process according to claim 6, wherein the cyclodextrin and the organic nitrate ester are used in equimolar amounts.

10. The process according to claim 6, wherein tert.-butyl methyl ether (TMBE) is used as asymmetric ether.

11. A 1:1 complex of an organic nitrate ester with a cyclodextrin prepared according to claim 6.

12. (canceled)

13. The complex according to claim 2, wherein the organic nitrate ester is glycerol trinitrate (GTN).
